(19) United States
(12) Patent Application Publication
(10) Pub. No.: US 2006/0281105 A1
(43) Pub. Date: Dec. 14, 2006

(54) High throughput multiplex DNA sequence amplifications

(76) Inventors: Honghua Li, Monmouth Junction, NJ (US); James Li, Monmouth Junction, NJ (US)

Correspondence Address: PERKINS COIE LLP, POST OFFICE BOX 1208, SEATTLE, WA, 98111-1208, US

(21) Appl. No.: 11/400,026

(22) Filed: Apr. 7, 2006

Related U.S. Application Data
(63) Continuation-in-part of application No. 10/530,544.
(60) Provisional application No. 60/417,009, filed on Oct. 7, 2002.

(51) Int. Cl. C12Q 1/68; G06F 19/00; C12P 19/34

(52) U.S. Cl. 435/6; 435/91.2; 702/20

(57) ABSTRACT

The present invention provides methods of designing PCR primers that allow the efficient and simultaneous amplification of a large number of different desired DNA fragments in a single multiplex PCR and minimize the formation of nonspecific extensions of undesired DNA fragments. The present invention allows a multiplex PCR to use at least 50 pairs of primers and produce at least 50 DNA fragments of interest. The present invention significantly broadens the application of multiplex PCR in the identification of multiple genes related to multifactorial diseases, the genome-scale detection of genetic alterations, the studies in large-scale pharmacogenetic reactions, the genotyping genetic polymorphism in a large population, the gene expression profiling in various samples, and high throughput genotyping technologies.

### CROSS-REFERENCE TO RELATED APPLICATIONS

The present application is a continuation-in-part of U.S. patent application Ser. No. 10/530,544, filed Apr. 7, 2005, which is a U.S. national phase application of PCT/US2003/031874, filed Oct. 7, 2003, which claims priority to U.S. Provisional Patent Application No. 60/417,009, filed Oct. 7, 2002, the disclosures of all of which are incorporated herein by reference as if fully set forth herein, including specification, drawings, and tables.

### REFERENCE TO GOVERNMENT GRANT

This invention is made with government support under grant R01-HG02094 awarded by the National Human Genome Research Institute. The U.S. government may have certain rights in this invention.

### FIELD OF THE INVENTION

This invention pertains to the field of high throughput multiplex DNA sequence amplification. Specifically, the invention pertains to methods of designing primers that allow the simultaneous amplification of a multiplicity of DNA fragments in a single polymerase chain reaction and minimize the formation of nonspecific extension of undesired DNA fragments.

### BACKGROUND

The polymerase chain reaction (PCR) is a primer-directed in vitro reaction for the enzymatic amplification of a specific DNA fragment. Saiki, Enzvmatic Amplification of β-Actin Genomic Sequences and Restriction Site Analysis for Diagnosis of Sickle Cell Anemia, Science 230: 1350-54 (1985). The PCR process is involved in the repetitive cycles of denaturation, primer annealing and extension by a thermostable DNA polymerase of two oligonucleotide primers that flank the DNA region of interest in a template DNA sample. At the beginning the PCR process, the duplex DNA target is denatured into two separated strands of DNA through a first heating step. In a subsequent annealing step, each oligonucleotide primer anneals or hybridizes to the complementary sequence of one separated strand of the target DNA. In a third extension step, nascent DNA is synthesized by extending each primer from its 3′ hydroxyl end of towards the 5′ end of the annealed target DNA strand by a thermostable DNA polymerase. The heating or denaturation step, the primer annealing step and the enzymatic extention step together constitute a single PCR cycle. If the newly synthesized DNA strand extends to or beyond the region complementary to the other primer, it serves as a primer annealing site and a template for extension in a subsequent PCR cycle. As a result, the repetitive PCR cycles give rise to the exponential accumulation of a specific DNA fragment whose termini are defined by the 5′ ends of the two primers. Theoretically, at the nth cycle of the PCR process, a single DNA molecule can produce 2ⁿ progeny DNA fragments of interest.

The distinctive nature of the PCR process in producing a substantive quantity of DNA fragments of interest from an initial tiny amount of DNA sample has gained broad applications in the field of biomedical research and clinical diagnosis. For example, PCR has been widely used in the diagnosis of inherited disorder and the individualization of evidence samples in the forensics area. Erlich et al, Recent Advances in the Polymerase Chain Reaction, Science 252: 1643-51 (1991); Newton & Graham, PCR (Oxford, 1994). In particular, PCR has played a critical role in genotyping a vast number of genetic polymorphisms and individual variations which underlie the onset of many diseases. Shi, Enabling Large-Scale Pharmacogenetic Studies by High-throughput Mutation Detection and Genotyping Technologies, Clin. Chem. 47: 164-172 (2001).

Widespread applications notwithstanding, the use of PCR is quite often limited by cost, time, and the availability of adequate test samples. To illustrate, the human genome project has placed over 6000 DNA markers in human genetic mapping. To analysis these 6000 markers in 1000 specimens, a total of 6,000,000 PCR reactions are needed if only one marker sequence is amplified in each reaction. As a well equipped laboratory may process 300 reactions and post-PCR assay a day, it will take a total of 20,000 working days or 80 years to complete the analysis, provided that the amount of each specimen suffices 6000 reactions.

In overcoming these limitations, a variant PCR termed multiplex PCR has been developed. Chamberlian et al, Deletion Screening of the Duchenne Muscular Dystrophy Locus via Multiplex DNA Amplification, Nucleic Acids Res. 16: 11141-56 (1988). Unlike the standard or uniplex PCR where only one pair of primers is used to amplify a single DNA fragment of interest, the multiplex PCR includes more than one pair of primers and thus results in more than one DNA fragment. Since its inception, the multiplex PCR has been applied in many areas of DNA testing, including gene deletion analysis, Chamberlain, supra, mutation and polymorphism analysis, Rithidech et al, Combining Multiplex and Touch Down PCR to Screen Murine Microsatellite Polymorphism, Bio-Techniques 23: 36-45 (1997), quantitative analysis, Zimmermann et al, Quantitative Multiple Competitive PCR of HIV-DNA in a Single Reaction Tube, BioTechniques 21: 480-484 (1996), RNA detection, Zou, Identification of New Influenza B virus Variants by Multiplex Reverse Transcription-PCR and the Heteroduplex Mobility Assay, J. Clin. Microbiol. 36: 1544-1548 (1998), and identification of microorganisms, Elnifro et al, Multiplex PCR: Optimization and Application in Diagnostic Virology, Clin. Microbiol. Rev. 13: 559-570 (2000).

Conceptually, the multiplex PCR has the potential to produce considerable savings in cost, time and sample volume. In aforementioned project of analyzing 6000 DNA markers in 1000 specimens, if n pairs of primers are used in a multiplex PCR reaction, it will only cost one-nth of 20,000 working days to complete the project as well as one-nth of the cost and sample volume required in the uniplex PCR reactions. Despite the attractive potential, the application of the multiplex PCR poses many challenges. For example, even under carefully optimized reaction conditions, only 26 DNA fragments could be amplified simultaneously in a single multiplex PCR. Edwards & Gibbs, Multiplex PCR: Advantages, Developments and Applications, PCR Meth. Appl. 3: S65-75 (1994); Lin et al, Multiplex Genotype Determination at a Large Number of Gene Loci, Proc. Natl. Acad. Sci. USA 93: 2582-2587 (1996).

Researchers are facing two tiers of challenge in optimizing the multiplex PCR. The first tier of challenge is the efficacy of PCR. In general, this issue is ubiquitous in all PCR reactions, whether in multiplex PCR or uniplex PCR. The efficacy of PCR is measured by its specificity, efficiency and fidelity. A highly specific PCR will generate one and only one amplified DNA fragment of intended sequence from each pair of primers. More efficient amplification will generate more products with fewer PCR cycles. A high-fidelity PCR product has the minimal amount of DNA polymerase-induced errors. Studies have shown the efficacy of PCR is affected by factors including the primer annealing temperature, the activity and concentration of the thermostable DNA polymerase, the PCR buffer components such as dNTPs and MgCl₂, and the first cycle set-up. Roux, Optimization and Troubleshooting in PCR, PCR methods Appl. 4: S185-S 194 (1995); Roberston & Walsh-Weller, An Introduction to PCR Primer Design and Optimization of Amplification Reactions, Methods Mol. Biol. 98: 121-154 (1998). Special attention has also been paid to the primer parameters, such as homology of primers with their target DNA sequence, primer length, GC content, ratio of primers to the template DNA. Researchers are cautioned that the efficacy of PCR is often a delicate balance among specificity, efficiency and fidelity. Cha & Thilly, Specificity, Efficiency, and fidelity of PCR, PCR Methods. Appl. 3: S 18-S 19 (1993). Adjusting the conditions for specificity may compromise the efficiency or fidelity and vise versa.

The second tier of challenge in multiplex PCR is the presence of multiple pairs of primers that are unique to multiplex PCR. It is reported that the presence of more than one primer pair increases the chance of obtaining spurious amplification products, primarily because of the formation of nonspecific DNA extensions, e.g., primer dimers. Markoulatos et al, Multiplex Polymerase Chain Reaction: A Practical Approach, J. Clin. Lab. Anal. 16: 47-51 (2002). The nonspecific extensions occur when 1) a first primer non-specifically interacts with a second primer because the first primer shares a certain degree of complementarity in its 3′ sequence with the 3′ sequence of the second primer; and 2) when a primer non-specifically interacts with a DNA sequence of a template DNA which is not the target DNA sequence. Elnifro, supra. The nonspecific extensions undermine not only the specificity of PCR but the efficiency as well. The nonspecific products compete with desired target DNA, consume the limited supplies of enzymes, primers and nucleotides, and produce impaired rates of annealing and extension. Markoulatos, supra. Not surprisingly, the non-specific extension limits the number of desired DNA fragments in a single multiplex PCR and poses a major limitation to the application and efficacy of multiplex PCR. Lin et al, Multiplex Genotype Determination at a Large Number of Gene Loci, Proc. Natl. Acad. Sci. USA 93: 2582-2587 (1996).

So far little progress has been made in combating the nonspecific extension problem. Researchers have developed a method to lower the chance of forming the nonspecific extension by adding a universal tail sequence to the 5′ end of the sequence-specific primers. Lin et al, supra; Brownie et al, The Elimination of Primer-Dimer Accumulation in PCR, Nucleic Acids Res. 25: 3235-3241 (1997). The tailed primers are added in a multiplex PCR reaction at very low concentrations and allowed to participate the early cycles of reaction. In subsequent cycles, the primers complementary to the universal tail sequence are added into the reaction at high concentrations and proceeded to continue PCR cycles. This method has reportedly produced 26 DNA fragments and minimized the accumulation of non-specific extensions. Lin et al, supra. However, the addition of a tail sequence does not thoroughly tackle the problem of non-specific interaction among primers or between a primer and a target DNA.

Thus, there is a need in the art to design primers that allow the simultaneous amplification of a multiplicity of DNA fragments in a single polymerase chain reaction. There is a need in the art to design primers that minimize or substantially reduce the formation of nonspecific extension of undesired DNA fragments. There is a need in the art to design primers that significantly enhance the efficacy of multiplex polymerase chain reactions.

### BRIEF DESCRIPTION OF THE DRAWINGS

FIG. 1 is an illustration of five forms of primer-primer interactions.

FIG. 2 is an illustration of three forms of interactions between primers and nonspecific target templates.

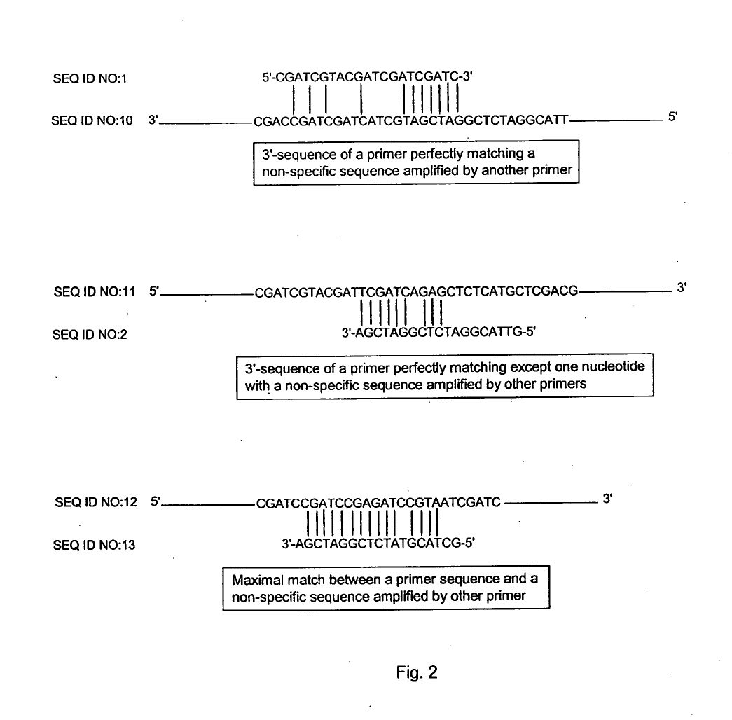

FIG. 3 is an illustration of a genotyping microarray determining the genotypes of a DNA sample at the 627 loci.

FIG. 4 is an illustration of a set of criteria in designing primers that are experimentally acceptable.

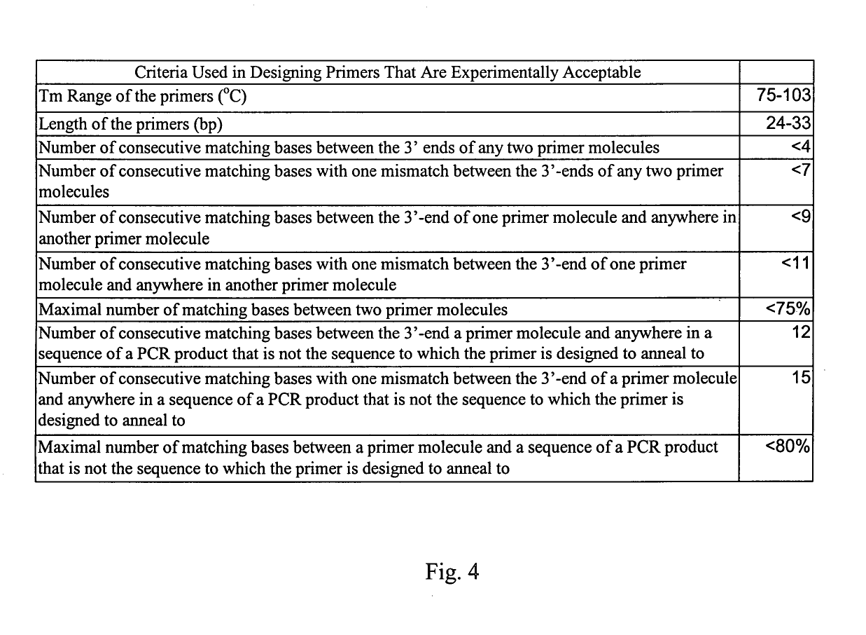

### SUMMARY OF THE INVENTION

One aspect of the present invention relates to methods of designing PCR primers that allow the efficient and simultaneous amplification of a large number of different desired DNA fragments in a single multiplex PCR and minimize the formation of nonspecific extensions of undesired DNA fragments.

In one embodiment of the invention, the method of designing primers to minimize the nonspecific extensions between a first primer and a second primer or the first primer comprises the steps of aligning the first primer and the second primer and selecting a first primer wherein:
1) the first primer at its 3′ end does not contain four or more bases that are perfectly matching to the 3′ end sequence of the first primer or a second primer;
2) the first primer at its 3′ end does not contain seven or more bases that are perfectly matching except one mismatch to the 3′ end sequence of the first primer or the second primer;
3) the first primer at its 3′ end does not contain six or more bases that are perfectly matching to a sequence anywhere of the first primer or the second primer;
4) the first primer at its 3′ end does not contain eleven or more bases that are perfectly matching except one mismatch to a sequence anywhere of the first primer or the second primer.
5) the maximal match between the first primer and the second primer does not exceed 75%.

In another embodiment of the invention, the method of designing primers to minimize the nonspecific extensions between a primer and a non primer-specific region of the a template DNA comprises the steps of aligning the primer and the template DNA and selecting a primer wherein:
1) the primer at its 3′ end does not contain 13 or more bases that are perfectly matching to any sequence of a DNA template other than the specific sequence to which the primer is. complementary; and 2) the primer at its 3′ end does not contain 17 or more bases that are perfectly matching except one mismatch to any sequence of a DNA template other than the specific sequence to which the primer is complementary.

In another embodiment of the invention, the method of designing primers to minimize the nonspecific extensions in a multiplex PCR comprises the steps of selecting a first primer wherein:
1) the first primer at its 3′ end does not contain four or more bases that are perfectly matching to the 3′ end sequence of the first primer or a second primer;
2) the first primer at its 3′ end does not contain seven or more bases that are perfectly matching except one mismatch to the 3′ end sequence of the first primer or the second primer;
3) the first primer at its 3′ end does not contain six or more bases that are perfectly matching to a sequence anywhere of the first primer or the second primer;
4) the first primer at its 3′ end does not contain eleven or more bases that are perfectly matching except one mismatch to a sequence anywhere of the first primer or the second primer;
5) the first primer at its 3′ end does not contain 15 or more bases that are perfectly matching to any sequence of a DNA template other than the specific sequence to which the primer is complementary;
6) the primer at its 3′ end does not contain 18 or more bases that are perfectly matching except one mismatch to any sequence of a DNA template other than the specific sequence to which the primer is complementary; and
7) the maximal match between the first primer and the second primer used inthemultiplex amplification does not exceed 75%.

Another aspect of the present invention relates to computer products or computer programs which, once executed by a computer process, perform methods as disclosed in the present invention.

The methods according to the present invention increase the number of desired DNA fragments, enhance the efficacy of the multiplex PCR and achieve a significant reduction in cost, time and sample volume. A single multiplex PCR using primers designed by the present invention can contain at least 50 pairs of primers and produce at least 50 desired DNA fragments.

The methods according to the present invention significantly broaden the application of multiplex PCR in the identification of multiple genes related to multifactorial diseases, the genome-scale detection of genetic alterations, the studies in large-scale pharmacogenetic reactions, the genotyping genetic polymorphism in a large population, the gene expression profiling in various samples, and high throughput genotyping technologies which include oligonucleotide ligation assay, pyrosequencing, single-base extension with fluorescence detection, homogeneous solution hybridization, molecular beacon genotyping, DNA chip-based microarray, and mass spectrometry technology.

### DETAILED DESCRIPTION OF THE INVENTION

The primary aspect of the present invention provides methods of designing PCR primers that allow the efficient and simultaneous amplification of a large number of different desired DNA fragments in a single multiplex PCR and minimize the formation of nonspecific extensions of undesired DNA fragments.

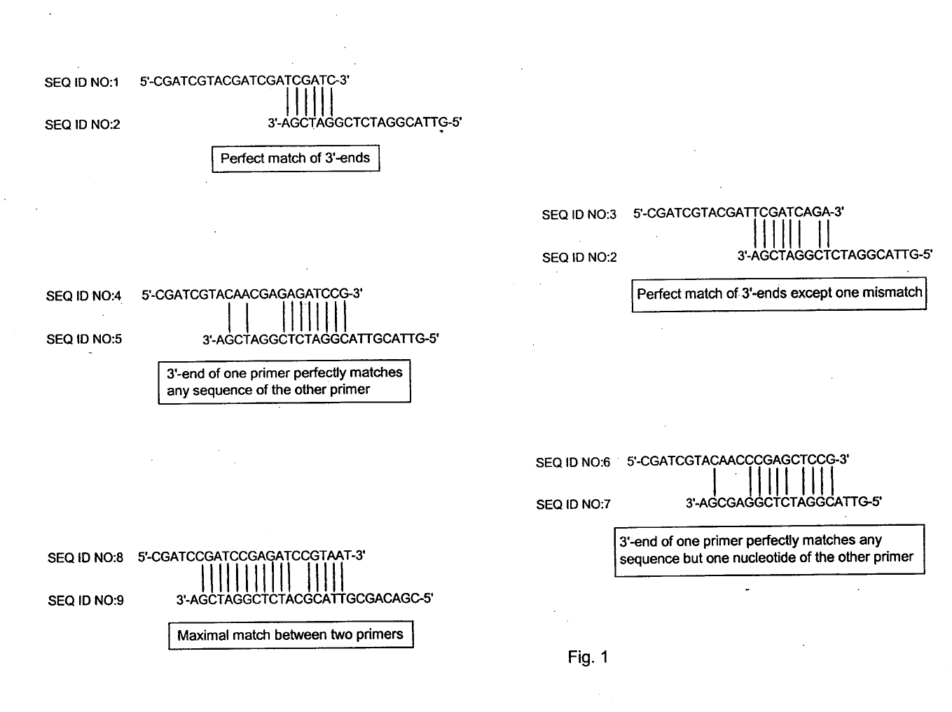

The nonspecific extension of unwanted DNA fragments is a major factor in preventing effective applications of multiplex PCR. The nonspecific extension is caused by nonspecific interactions between different molecules of either the same primer, or different primers, or a primer and a non-primer specific region of DNA templates. Specifically, the nonspecific interactions are caused by 1) a stretch of perfectly matched sequence at the 3′ ends of two primers, 2) a stretch of perfectly matched sequence with only one mismatch at the 3′ ends of two primers, 3) a stretch of the 3′ end sequence of a primer perfectly matching to the internal sequence of the same primer, another primer, or a non-primer specific region of a DNA template, 4) a stretch of the 3′ end sequence of a primer perfectly matching with only one mismatch to the internal sequence of itself, another primer, or a non-primer specific region of a DNA template, or 5) a stretch of a sequence in a primer matching to itself, another primer, or a non-primer specific region of a DNA template. FIG. 1. The nonspecific extensions of undesired DNA fragments compete and consume the same reagent components in the PCR reaction and thus impair the production and extension of desired DNA fragments. The problem of nonspecific extensions is aggravated when multiple pairs of primers of high concentrations are present in a single multiplex PCR.

One embodiment of the present invention circumvents the nonspecific extension by setting forth a list of criteria in designing PCR primers useful for multiplex PCR. According to one embodiment of the invention, the method of designing primers to minimize the nonspecific extensions between a primer and a all the rest of primers including the primer comprises the steps of selecting a first primer wherein:
1) the first primer at its 3′ end does not contain four or more bases that are perfectly matching to the 3′ end sequence of the first primer or a second primer;
2) the first primer at its 3′ end does not contain seven or more bases that are perfectly matching except one mismatch to the 3′ end sequence of the first primer or the second primer;
3) the first primer at its 3′ end does not contain six or more bases that are perfectly matching to a sequence anywhere of the first primer or the second primer; and
4) the first primer at its 3′ end does not contain eleven or more bases that are perfectly matching except one mismatch to a sequence anywhere of the first primer or the second primer.

The same method repeatedly applies to the selection of a subsequent primer until all the selected primers meet the above criteria.

According to another embodiment of the invention, the method of designing primers to minimize the nonspecific extensions between a primer and a non primer-specific region of the a template DNA comprises the steps of selecting a primer wherein:
1) the primer at its 3′ end does not contain 13 or more bases that are perfectly matching to any sequence of a DNA template other than the specific sequence to which the primer is complementary; and
2) the primer at its 3′ end does not contain 17 or more bases that are perfectly matching except one mismatch to any sequence of a DNA template other than the specific sequence to which the primer is complementary.

According to another embodiment of the invention, the method of designing primers to minimize the nonspecific extensions in a multiplex PCR comprises the steps of selecting a first primer wherein: 1) the first primer at its 3′ end does not contain four or more bases that are perfectly matching to the 3′ end sequence of the first primer or a second primer; 2) the first primer at its 3′ end does not contain seven or more bases that are perfectly matching except one mismatch to the 3′ end sequence of the first primer or the second primer;
3) the first primer at its 3′ end does not contain six or more bases that are perfectly matching to a sequence anywhere of the first primer or the second primer;
4) the first primer at its 3′ end does not contain eleven or more bases that are perfectly matching except one mismatch to a sequence anywhere of the first primer or the second primer,
5) the first primer at its 3′ end does not contain 13 or more bases that are perfectly matching to any sequence of a DNA template other than the specific sequence to which the primer is complementary; and
6) the primer at its 3′ end does not contain 17 or more bases that are perfectly matching except one mismatch to any sequence of a DNA template other than the specific sequence to which the primer is complementary.

In practicing the present invention, each primer to be used in a multiplex PCR is selected through the methods described herein. The selection of primers for a large number of DNA templates can be conducted manually or through a computer system. In a preferred embodiment, the methods according to the present invention are conducted through the use of a computer system.

A computer system according to the present invention refers to a computer or a computer readable medium designed and configured to perform some or all of the methods as described herein. A computer used herein may be any of a variety of types of general-purpose computers such as a personal computer, network server, workstation, or other computer platform now or later developed. As commonly known in the art, a computer typically contains some or all the following components, for example, a processor, an operating system, a computer memory, an input device, and an output device. A computer may further contain other components such as a cache memory, a data backup unit, and many other devices. It will be understood by those skilled in the relevant art that there are many possible configurations of the components of a computer.

A processor used herein may include one or more microprocessor(s), field programmable logic arrays(s), or one or more application specific integrated circuit(s). Illustrative processors include, but are not limited to, Intel Corp's Pentium series processors, Sun Microsystems' SPARC processors, Motorola Corp.'s PowerPC processors, MIPS Technologies Inc.'s MIPs processors, and Xilinx Inc.'s Vertex series of field programmable logic arrays, and other processors that are or will become available.

A operating system used herein comprises machine code that, once executed by a processor, coordinates and executes functions of other components in a computer and facilitates a processor to execute the functions of various computer programs that may be written in a variety of programming languages. In addition to managing data flow among other components in a computer, an operating system also provides scheduling, input-output control, file and data management, memory management, and communication control and related services, all in accordance with known techniques. Exemplary operating systems include, for example, a Windows operating system from the Microsoft Corporation, a Unix or Linux-type operating system available from many vendors, any other known or future operating systems, and some combination thereof.

A computer memory used herein may be any of a variety of known or future memory storage devices. Examples include any commonly available random access memory (RAM), magnetic medium such as a resident hard disk or tape, an optical medium such as a read and write compact disc, or other memory storage devices. A memory storage device may be any of a variety of known or future devices, including a compact disk drive, a tape drive, a removable hard disk drive, or a diskette drive. Such types of memory storage device typically read from, and/or write to, a computer program storage medium such as, respectively, a compact disk, magnetic tape, removable hard disk, or floppy diskette. Any of these computer program storage media, or others now in use or that may later be developed, may be considered a computer program product. As will be appreciated, these computer program products typically store a computer software program and/or data. Computer software programs, also called computer control logic, typically are stored in system memory and/or the program storage device used in conjunction with memory storage device.

In one embodiment, a computer program product as described herein comprising a computer memory having a computer software program stored therein, wherein the computer software program when executed by a processor or in a computer performs methods according to the present invention.

An input device used herein may include any of a variety of known devices for accepting and processing information from a user, whether a human or a machine, whether local or remote. Such input devices include, for example, modem cards, network interface cards, sound cards, keyboards, or other types of controllers for any of a variety of known input function. An output device may include controllers for any of a variety of known devices for presenting information to a user, whether a human or a machine, whether local or remote. Such output devices include, for example, modem cards, network interface cards, sound cards, display devices (for example, monitors or printers), or other types of controllers for any of a variety of known output function. If a display device provides visual information, this information typically may be logically and/or physically organized as an array of picture elements, sometimes referred to as pixels.

As will be evident to those skilled in the relevant art, a computer software program of the present invention can be executed by being loaded into a system memory and/or a memory storage device through one of the above input devices. On the other hand, all or portions of the software program may also reside in a read-only memory or similar type of memory storage device, such devices not requiring that the software program first be loaded through input devices. It will be understood by those skilled in the relevant art that the software program or portions of it may be loaded by a processor in a known manner into a system memory or a cache memory or both, as advantageous for execution.

As will be appreciated by those skilled in the art, a computer program product of the present invention, or a computer software program of the present invention, may be stored on and/or executed in a PCR instrument. For example, a computer software of the present invention can be installed in, for example, the Smart Cycler System, the Idaho Rapid Cycler, the Carbett Roter-Gene System, the GeneAmp 5700 Sequence Detection System, the ABI Prism7000, 7700 & 7900 Sequence Detection Systems, the icycler System, the MX-4000 Multiplex Quantitative PCR System, the DNA Engine Opticon System, the Perkin-Elmer 9600 cycler, and MJ Research's DNA Engine Opticon System.

However, it is not necessary that the computer program product or the computer software program be stored on and/or executed in a PCR instrument. Rather, the computer product or software may be stored in a separate computer or a computer server which may or may not connect to the PCR instrument through a data cable, a wireless connection, or a network system. As commonly known in the art, network systems comprise hardware and software to electronically communicate among computers or devices. Examples of network systems may include arrangement over any media including Internet, Ethernet 10/1000, IEEE 802.11x, IEEE 1394, xDSL, Bluetooth, 3G, or any other ANSI approved standard.

In a preferred embodiment, a computer program termed MULTIPLEX is developed to select primers according to the methods as described in the present invention. See Table I for the flowchart of MULTIPLEX program.

Even with the assistance of MULTIPLEX, it is time consuming to analyze exhaustively all possible sequences frames and select the best possible frames for PCR primers. To expedite the computer-assisted selection process, a strategy termed “random fitting” is developed. Under the random fitting strategy, a set of criteria for the length of the matching sequences is set forth for primer selection. See Table I. For example, when the number of 3′ end matching bases is less than 4, the experimental effect of this complementarity is neglected. Therefore, the criterion for the length of 3′ end complementarity was set to be less than four. With the predefined criteria, the MULTIPLEX computer program first randomly picks up a pair of primers for each target sequence. All possible interacting pairs in this combination are examined. Record is made on qualified and unqualified primers in the combination. The program then randomly picks up a new pair of primers for each target sequence that collectively form a second combination. If the number of qualified primers in the second combination is less than that in the first combination, no record is made. The MULTIPLEX program, however, begins to examine a third combination. If.the number of qualified primers in the third combination is greater than that in the first combination, the first primer combination is replaced by the third one in record. The program keeps processing until a combination with all qualified primers is found. Under the random fitting strategy, the MULTIPLEX program can select qualified primers for 100 sequences within two hours, 500 within two days and 1,000 within two weeks. The “qualified primers” are those primers fully conforming with the selection criteria set forth in the method of the present invention.

To further improve the MULTIPLEX program, another primer selection method called linear primer selection is also used as an alternative. See Table I. With this strategy, instead of selecting the frames randomly, each frame of a pair is selected from one end of the defined range of a sequence. The selected frame pair is then examined. If these frames are qualified as primer sequences, the selection of primers for the corresponding sequence is completed. Otherwise, the selection will be continued by sliding the frames by one base toward the other ends of the sequences. The newly selected frames are then examined. If these frames are qualified as primer sequences, the selection of primers for the corresponding sequence is then completed. Otherwise, the selection will be continued by sliding the frames by one base toward the other ends of the sequences... If the frames are slid to the other ends but not qualified frames are found, the lengths of the frames with be increased by 1 base. The same process described above will be repeated. The sliding and length changing process repeats until a pair of qualified frames is found. If no qualified frames can be found after exhausting all possible frames for a sequence, the sequence will be labeled as unusable, and will be excluded from the multiplex set. This method is called linear primer selection.

When the number of sequences is large, the random primer selection method may be used for selecting primers of only a fraction of sequences. The random selection process is stopped at a point defined by the user. The program can then switch to linear primer selection method. We have shown that appropriate combination of these two methods can increase the selection speed by several tens to >100 fold compared with using the random method only.

It needs to be pointed out that the MULITPLEX method can be used not only for primer selection of SNPs, but also for primer selection of any other DNA and RNA sequences if a position is defined so that it can be used to separate a sequence into two parts for selecting the two primers, respectively.

Following the selecting and synthesizing of qualified primers, DNA templates are contacted with multiple primers for the amplification of desired DNA fragments under conditions suitable for multiplex PCR developed in the inventor's laboratory. These conditions are: 2.0 mM MgCl₂, 50 mM KCl, 100 mM Tris-HCl, pH 8.3, 100 μM deoxynucleotide triphosphates (dNTPs), and 10 units/50 μl “HotStart” Taq DNA polymerase (Qiagen, Valencia Calif.). The PCR mix is first preheated for 15 min at 94° C. to activated the DNA polymerase followed by 40 PCR cycles. Each cycle consists of a denaturation step at 94° C. for 40 sec, and then an annealing step at 55° C. for 2 min followed by a ramping step from 55° C. to 70° C. within 5 min. After the PCR cycles, the samples are incubated at 72° C. for 3 min.

A DNA template to be used in practicing the present invention includes without limitation eukaryotic, prokaryotic and viral DNA. The DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. DNA is extracted from the cell source or body fluid using any of the numerous methods that is standard in the art. It will be understood that the particular method used to extract DNA will depend on the nature of the source. The preferred amount of DNA to be extracted for use in the present invention is at least 5 pg which is corresponding to about 1 human cell equivalent of a genome size of 4×10⁹ base pairs.

A primer designed in accordance to the method in the present invention is from 17 to 50 nucleotides in length, preferably 20 to 35 nucleotides in length. The concentration of a primer in the multiplex PCR reaction can range from 0.1 nM to about 4 μM per reaction, preferably from lnM to 0.1 4 μM per reaction.

Multiplex PCR reactions are carried out using manual or automatic thermal cycling. Any commercially available thermal cycler may be used, such as, e.g., a Perkin-Elmer 9600 cycler.

The resultant multiple amplified DNA fragments of interest are analyzed using any of several methods that are well-known in the art. For example, agarose or polyacrylamide gel electrophoresis is used to rapidly resolve and identify each of the amplified sequences. When a gel is used, different amplified sequences are preferably of distinct sizes and thus can be resolved in a single gel. The reaction mixture can further be treated with one or more restriction endonucleases prior to electrophoresis. Alternative Methods of product analysis include without limitation dot-blot hybridization with allele-specific oligonucleotides, single-strand conformational polymorphism analysis, high-througput genotyping platforms including oligonucleotide ligation assay, pyrosequencing, single-base extension with fluorescence detection, homogeneous solution hybridization, molecular beacon genotyping, DNA chip-based microarray, and mass spectrometry technology.

The multiple primers designed in accordance to the method in the present invention minimize the nonspecific interaction between primers or between a primer and nonspecific target sequence of a template DNA. Accordingly, the use of these primers in a multiplex PCR minimizes the formation of non-specific extension of undesired DNA fragments and maximizes the specific interaction and amplification of desired DNA fragments. Furthermore, the method in the present invention increases the number of desired DNA fragments, enhances the efficacy of the multiplex PCR and achieves a significant reduction in cost, time and sample volume. Finally, the multiple primers designed in accordance with the methods of the present invention may be used in real time PCR or multiplex real time PCR.

A single multiplex PCR using primers designed by the present invention can contain at least 50 pairs of primers and produce at least 50 desired DNA fragments. It is preferred that the single multiplex PCR contain at least 100 pairs of primers and produce at least 100 desired DNA fragments.

The present invention significantly broadens the application of multiplex PCR in the art which has been limited by the nonspecific extensions of unwanted DNA fragment and the number of desired DNA fragments it could produce. Given a large number of multiple desired DNA fragments that a multiplex PCR now can produce using primers designed under the present invention, the multiplex PCR can now be fully used in applications including but not limited to the identification of multiple genes related to multifactorial diseases, the genome-scale detection of genetic alterations in cancers, the studies in large-scale pharmacogenetic reactions, the genotyping genetic polymorphism in a large population, and the gene expression profiling in various samples.

The following examples are intended to further illustrate the present invention without limiting the invention thereof.

### EXAMPLE 1

### Selection of 627 Pairs of Primers

648 single nucleotide polymorphism (SNP) markers were initially selected from the SNP Database maintained by the National Center for Biotechnology Information. To facilitate the genotyping after PCR, all these SNPs were transition polymorphisms that were A to G or C to T changes at their polymorphic sites. All SNP sequences were analyzed by the computer program MULTIPLEX to determine whether these SNP sequences are unique in the genome. The repetitive sequences were discarded. PCR primers were selected by using the computer program MULTIPLEX described above with the following values: Tₘ range=75-104° C., primer length range=24-33 bases, 3′ perfect matches <4, 3′ match with 1 mismatch <7, 3′ end matching internal sequences of other molecules <9; 3′ end matches internal sequences of other molecules with 1 mismatch <11; maximal match between different molecules, 75%). The quality of each pair of primers was examined individually by using them to amplify their target sequences. Only the primer pairs with high specificity and yield, as judged by gel electrophoresis, were used for multiplex amplification. At the end, a panel of 627 SNPs was selected from the initial 648 SNPs.

### EXAMPLE 2

### Using 622 Pairs of Selected Primers in a Single Multiplex PCR

For the multiplex PCR, lysate for 500 cells from a tissue cultured cell line, MG2314, was prepared. The reason for using cells instead of purified DNA is that they could be precisely quantified and equal number of nearly equal number of copies of the target sequences could be used as the starting material. PCR mix contained 1 X PCR buffer (100 mM Tris-HCl pH 8.3, 150 mM KCl, 1.5 mM MgCl₂, and Gelatin 100 μg/ml), primers (10 nM each) for all SNPs, the four dNTPs (100 μM each), Taq DNA polymerase (5 units) with a final volume of 30 μl. Sample was preheated for 15 min at 95° C. Each PCR cycle consisted of a denaturation step at 95° C. for 40 sec; annealing at 55° C. for 3 min; and a step for both annealing and extension with temperature ramping from 55° C. to 70° C. within 5 min. A 3 min incubation at 95° C. as added after the PCR cycle to minimize the incompletely extended PCR products. PCR was completed after 40 cycles.

### EXAMPLE 3

### Analysis of Multiple DNA Fragments After the Multiplex PCR

To resolve the allelic products in the multiplex PCR product for genotype determination, single base extension and microarray methods were used. Two oligonucleotides with completely complementary sequences for each SNP were synthesized for this purpose. One of these was called E probe that was using in the single base extension assay. The other was called A probe that was spotted onto a coated glass slide. E probes had sequences with their 3′-ends next to their polymorphic sites. In the single base extension assay, dideoxynucleotides labeled with either the chromaphore Cy 3 or Cy 5 were used. The allelic base at the polymorphic site determined which fluorescently labeled nucleotide could be incorporated into an E probe.

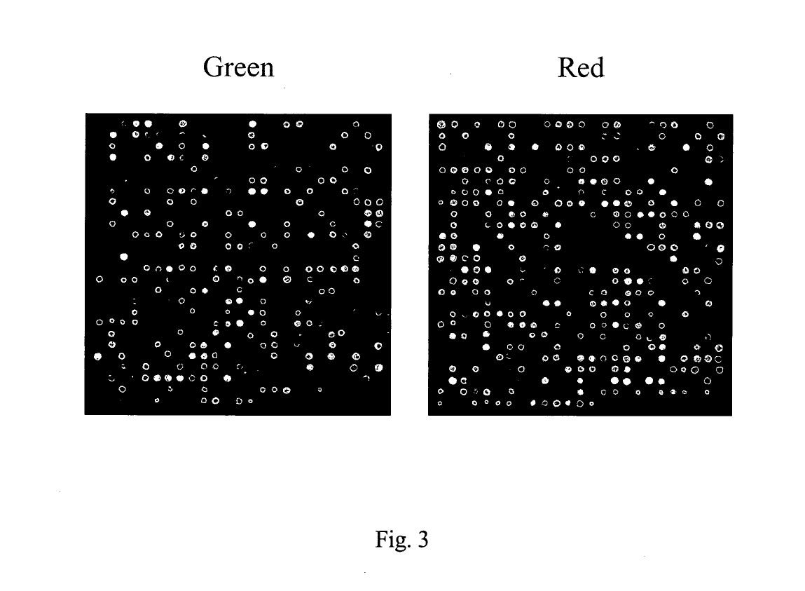

The corresponding A probes were spotted onto a glass slide with a microarrayer manufactured by Cartesian. The fluorescently labeled E probes were hybridized with the A probes on the microarray. The signal intensity for the alleles of each SNP was determined by using the computer software for image analysis from Biodiscovery. See, FIG. 3.

To validate the results from microarray analysis, the genotypes of the cell line used in the study were determined for all 622 SNPs by restriction enzyme digestion method described by Li & Hood, Multiplex Genotype Determination at A DNA Sequence Polymorphism Cluster in The Human Immunoglobulin Heavy-Chain Region, Genomics 26: 199-206 (1995). A few SNPs that could not be analyzed by this method were analyzed by direct sequence analysis.

Because all SNP were transition polymorphisms, all E probes could be analyzed by either A and G or T and C. In either case, consistent results from 85% (for labeling with A and G) to 90% (for labeling with T and C) SNPs were obtained by both microarray and the restriction digestion methods. A probes for A and G labeling were used for 85% of SNPs, and others were replaced by those for T and C labeling. FIG. 5.

Papers and patents listed in the disclosure are expressly incorporated by reference in their entirety. It is to be understood that the description, specific examples, and figures, while indicating preferred embodiments, are given by way of illustration and exemplification and are not intended to limit the scope of the present invention. Various changes and modifications within the present invention will become apparent to the skilled artisan from the disclosure contained herein. Therefore, the spirit and scope of the appended claims should not be limited to the description of the preferred versions contained herein.

TABLE I
Flow Chart - General

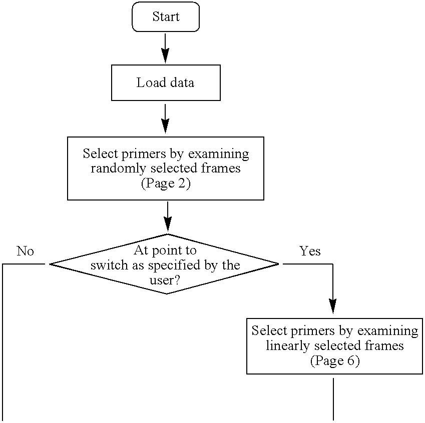

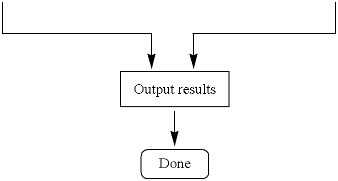

Page 2 - Select Primers by Examining
Randomly Selected Frames

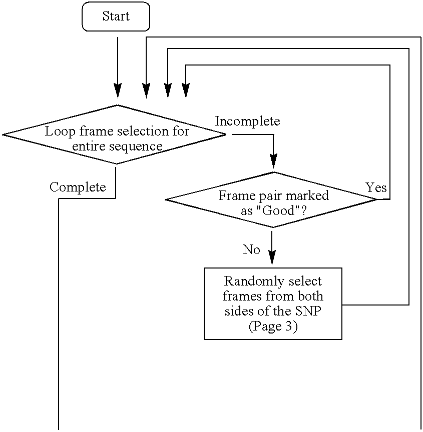

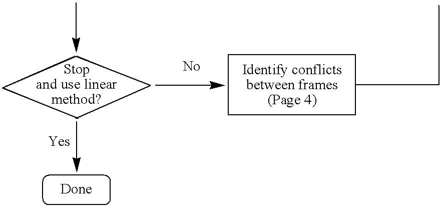

Page 3 - Randomly Select Frames from
Both Sides of Each SNP

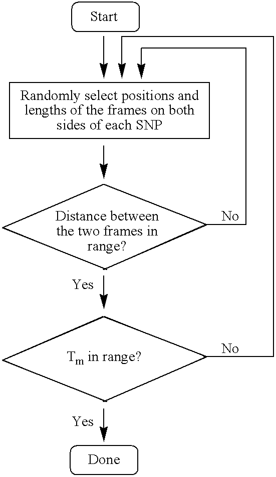

Page 4 - Identify Conflicts between Frames

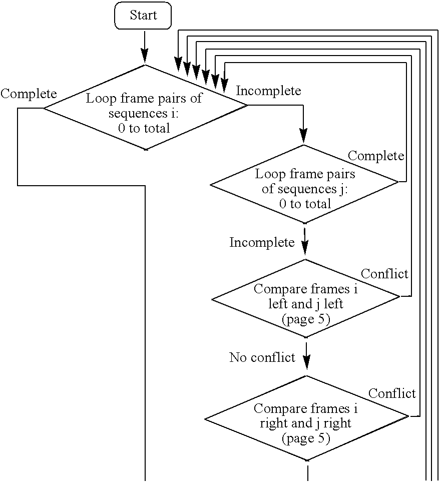

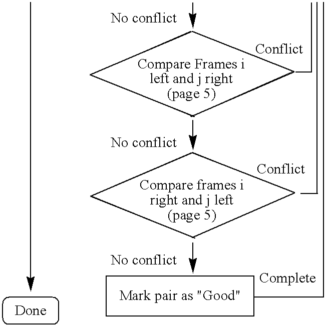

Page 5 - Compare Selected Frames

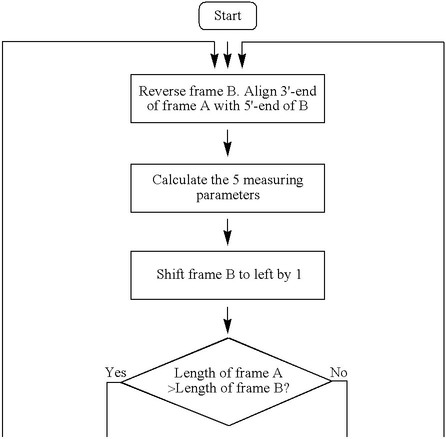

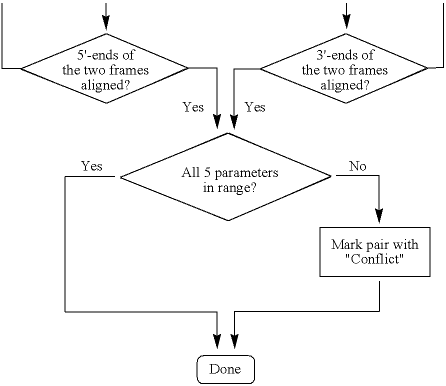

Page 6 - Select Primers by Examining
Linearly Selected Frames

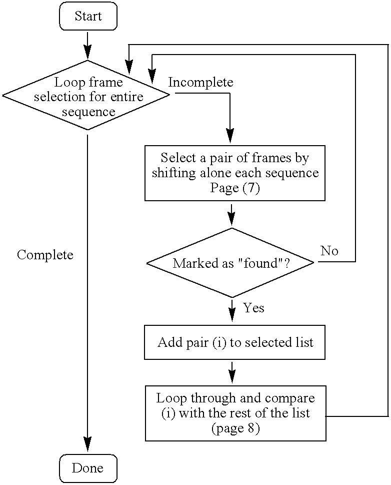

Page 7 - Select a pair of frames by shifting alone each sequence

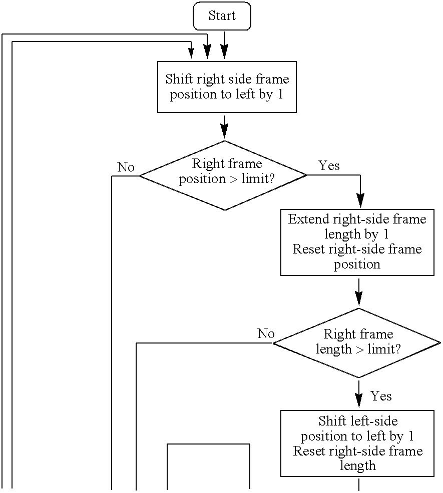

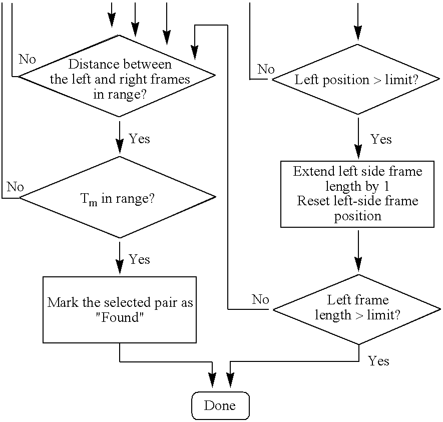

Page 8 - Loop through and Compare Frames i
with the Rest of the List

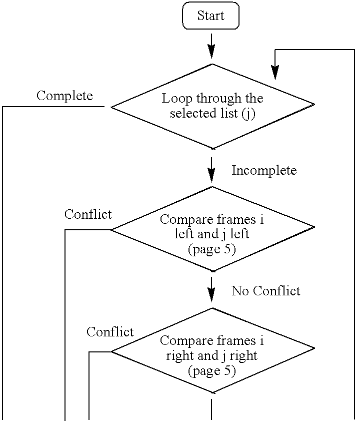

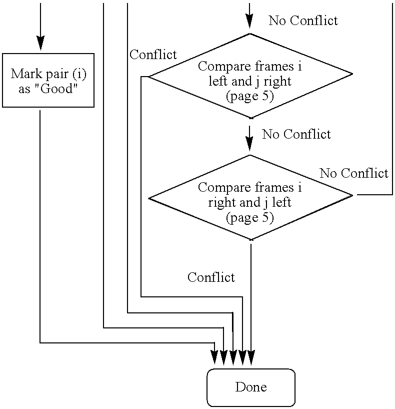

What is claimed is:

1. A method for designing a multiplicity of primers for simultaneous amplification of a multiplicity of target DNA fragments in a single multiplex polymerase chain reaction comprising the steps of:
a. aligning a first primer and a second primer; and
b. selecting the first primer wherein 1) the first primer at its 3′ end does not contain four or more bases that are perfectly matching to the 3′ end sequence of the first primer or a second primer; the first primer at its 3′ end does not contain seven or more bases that are perfectly matching except one mismatch to the 3′ end sequence of the first primer or the second primer; the first primer at its 3′ end does not contain six or more bases that are perfectly matching to a sequence anywhere of the first primer or the second primer; and the first primer at its 3′ end does not contain eleven or more bases that are perfectly matching except one mismatch to a sequence anywhere of the first primer or the second primer.

2. A method of claim 1 wherein at least 100 primers are designed.

3. A method of claim 2 wherein at least 200 primers are designed.

4. A method of claim 3 wherein at least 1000 primers are designed.

5. A method of claim 1 wherein at least 50 target DNA fragments are produced in the single multiplex polymerase chain reaction.

6. A method of claim 1 wherein at least 100 target DNA fragments are produced in the single multiplex polymerase chain reaction.

7. A method of claim 1 wherein at least 500 target DNA fragments are produced in the single multiplex polymerase chain reaction.

8. A method of claim 1 wherein the single multiplex polymerase chain reaction is used for an application.

9. A method of claim 8 wherein the application is selected from the group consisting of an identification of multiple genes related to multifactorial diseases, a genome-scale detection of genetic alterations in cancers, a study in large-scale pharmacogenetic reactions, a genotyping genetic polymorphism in a large population, and a gene expression profiling.

10. A method of claim 1 wherein the primers increase the efficacy of the single multiplex polymerase chain reaction.

11. A method of claim 1 wherein the primers minimize the non-specific extension of the single multiplex polymerase chain reaction.

12. A computer product comprising a computer readable medium containing a computer program which once executed by a computer processor performs the method of claims.


SEQUENCE LISTING (13 sequences)

SEQ ID NO: 1 - DNA, 21 bp, Artificial
  PCR primer
cgatcgtacgatcgatcgatc

SEQ ID NO: 2 - DNA, 19 bp, Artificial
  PCR primer
gttacggatctcggatcga

SEQ ID NO: 3 - DNA, 21 bp, Artificial
  PCR primer
cgatcgtacgattcgatcaga

SEQ ID NO: 4 - DNA, 22 bp, Artificial
  PCR primer
cgatcgtacaacgagagatccg

SEQ ID NO: 5 - DNA, 24 bp, Artificial
  PCR primer
gttacgttacggatctcggatcga

SEQ ID NO: 6 - DNA, 22 bp, Artificial
  PCR primer
cgatcgtacaacccgagctccg

SEQ ID NO: 7 - DNA, 19 bp, Artificial
  PCR primer
gttacggatctcggagcga

SEQ ID NO: 8 - DNA, 23 bp, Artificial
  PCR primer
cgatccgatccgagatccgtaat

SEQ ID NO: 9 - DNA, 26 bp, Artificial
  PCR primer
cgacagcgttacgcatctcggatcga

SEQ ID NO: 10 - DNA, 36 bp, Artificial
  PCR template
ttacggatctcggatcgatgctactagctagccagc

SEQ ID NO: 11 - DNA, 37 bp, Artificial
  PCR template
cgatcgtacgattcgatcagagctctcatgctcgacg

SEQ ID NO: 12 - DNA, 28 bp, Artificial
  PCR template
cgatccgatccgagatccgtaatcgatc

SEQ ID NO: 13 - DNA, 19 bp, Artificial
  PCR primer
gctacgtatctcggatcga